(19) United States
(12) Patent Application Publication
(10) Pub. No.: US 2025/0354021 A1
(43) Pub. Date: Nov. 20, 2025

(54) Paint comprising microbial cells and water-based paint vehicle

(71) Applicants: Carrine Blank, Missoula, MT (US); Nancy W. Hinman, Missoula, MT (US)

(72) Inventors: Carrine Blank, Missoula, MT (US); Nancy W. Hinman, Missoula, MT (US)

(21) Appl. No.: 18/666,978

(22) Filed: May 17, 2024

(51) Int. Cl. C09D 7/41; C09D 7/63; C09D 105/00; C12N 1/12

(52) U.S. Cl. CPC C09D 7/41; C09D 7/63; C09D 105/00; C12N 1/12

(57) ABSTRACT

This invention is an artist paint comprising a colorant and a water-based paint vehicle. The colorant comprises whole microalgal or cyanobacterial cells, which are grown under controlled light conditions comprising changes in light intensity or color for specified periods of time to produce the desired colorant. The water-based paint vehicle comprises a humectant, photostabilizers and preservatives, a buffer, and rheology modifiers. The paint is made by growing and harvesting the cells as the colorant followed by mixing with the water-based paint vehicle to create a paint suspension. The resulting suspension is allowed to dehydrate to form a moist cake. The cake is mixed with the water-based paint vehicle using a paint brush to create a second paint suspension that is then painted onto a surface. The resulting paint can be controlled to provide a uniform or a gritty texture.

### PRIOR ART

At least one specification heading is required. Please delete this heading section if it is not applicable to your application. For more information regarding the headings of the specification, please see MPEP 608.01(a).

### BACKGROUND OF THE INVENTION

### Uses

Colorants (defined as substances that impart color to matter, e.g., pigments) are used in dyes, inks, and paints. They are used for printing, dyeing, and painting. Colorants are used to color textiles, decorations, art, and food. Colorants are also used in business for printing documents, labels, and images, and they are used in industry to create coatings with specific properties.

In general, a dye is a solution of dissolved (soluble) colorant(s) that is used to color textiles, decorations, art, and food. Inks are thin suspensions of solid colorants (i.e. pigments), typically mixed with a liquid vehicle, that are used for the same purposes as dyes and also are used in business to print documents, labels, patterns, etc. Paints are thicker suspensions or emulsions of colored solids (i.e. pigments) used to create a dried colored film that is then used for decorations and art or as a protective and colorful coating for other purposes (e.g., structures, automotive). Paints comprise solids that are dispersed in a binder or vehicle, whereas inks (or dyes) are often pigment compounds (such as carbon black) that are dissolved in a solvent.

We invented a water-based paint made using natural pigments from whole, single-celled organisms (e.g., algae, cyanobacteria) as the colorant. The paint is photostable because the pigments are retained in whole cells and by virtue of the composition of the water-based paint vehicle (WBPV) (Art FIG. 1). The invention described herein reduces greenhouse-gas, environmental, and health impacts by using cultivated microalgal and cyanobacterial cells to provide the pigments.

### Sources of Pigments

Pigments provide color in the visible region (400-700 nanometers) of the light spectrum. They may be of natural biotic (living organisms) or abiotic origin (i.e. inorganic chemicals, minerals) or may be manufactured synthetically. They may be mixed with water or organic solvents to be used as dyes, inks, or paints.

### Toxic, Rare, and Environmentally Costly Components

At present, many commercially mass-produced water-based artist paints (i.e. watercolor or acrylic paints) are made with heavy metal compounds as pigments or synthetic organic pigments that are suspended in an aqueous paint vehicle. The heavy metals used in paints include salts of aluminum, calcium, copper, manganese, cadmium, cobalt, lithium, zinc, titanium, iron, lead, and silver. Some heavy metal salts (e.g., cadmium, cobalt, lead) are known to cause cancer or neurological disease. Some synthetic organic compounds used as pigments have safety profiles that may be poorly characterized and have carbon footprints that may be significant. Artists for years have expressed concerns about using paints that contain toxic chemicals.

There is a need for alternative, water-based paints that are less toxic or non-toxic, environmentally friendly, and sustainably sourced that do not compete for scarce resources. There is a need for an alternative way to make water-based paints that does not use heavy metals or synthetic compounds as pigments. There is also a need for an alternative way to make paints that have a lower carbon footprint. There is a need for a way to generate paints domestically that relies less upon international trade agreements. There is also a need to generate paints that do not compete with other industries that heavily rely on heavy metal mining, such as the semiconductor and superconductor industries.

Heavy metal minerals are also in high demand to make wind generators, produce solar power, and manufacture batteries for electronics and green technologies. Competition with multiple industries elevates the price of metals and limits their availability for use in paints. Their availability can also be subject to political instability. According to the USGS, the United States currently imports almost 50% of the minerals it consumes. This includes minerals refined to produce the heavy metals used in current watercolor paints. The minerals are mined as low-grade or high-grade ores and then purified and concentrated using complex chemical processes. Environmental regulations can be lax across the globe, exposing mine and factory workers to hazardous toxic chemicals. The purified heavy metals are then shipped worldwide. Consequently, paints made with heavy metals can have very large carbon and environmental footprints. For example, rare earth metals, used in some high-quality paints, are almost exclusively mined and processed (58%) and/or smelted (95%) in China; the US, Myanmar, and Australia represent less than 42% combined (e.g., Zapp, P., Schreiber, A., Marx, J. and Kuckshinrichs, W., 2022. Environmental impacts of rare earth production. MRS bulletin, 47(3), pp. 267-275). Cobalt, a strategic metal and a component of Cobalt Blue or Parrish Blue (artist water-based paint colors), is mined almost exclusively in the Democratic Republic of Congo and is shipped globally. As of 2020, 6.4% of global cobalt production is used for pigments (STATISTA. Distribution of cobalt demand worldwide in 2022, by application. https://www.statista.com/statistics/1143399/global-cobalt-consumption-distribution-by-application/, accessed Nov. 13, 2023). Consequently, a domestic source of pigments would reduce the competition with green technologies for metals, be more environmentally sustainable, and have a lower carbon footprint. Our paint meets this need by substituting existing mineral or synthetic organic pigments with organic microalgal and cyanobacterial pigments.

Historically, natural organic colorants have been derived from pigments isolated from vascular plants (e.g. indigo, woad, coffee, tea, curry powders), insects (e.g., cochineal) or cuttlefish (e.g., sepia). More recently a number of patents or patent applications apply pigments isolated or extracted from macroalgae (macroscopic photosynthetic eukaryotic organisms), microalgae (microscopic photosynthetic eukaryotic organisms), and/or cyanobacteria for use as dyes or inks (e.g., Venil, C. K., Zakaria, Z. A. and Ahmad, W. A., 2013. Bacterial pigments and their applications. Process Biochemistry, 48(7), pp. 1065-1079; Patel, Alok. https://www.linkedin.com/feed/update/urn: li: activity: 7196804391427862529/; US20200095729A1; U.S. Pat. No. 11,577,962; WO2021156462A1; JP2005295829A). These colorants are used as natural colorants for fabrics, food, or beverages. Many such pigments from natural organisms are intended for use in the food industry. Most biologically derived pigments are hydrophobic or are readily oxidized. Thus, in order to make the pigments stable and soluble in aqueous solutions, preservatives, detergents, and/or organic solvents are commonly employed to solubilize the pigments, and, in some cases, the resulting solution is heated to increase stability. For example, marine macroalga was used to dye mordant-treated silk by way of grinding and extracting the pigment using water and a heating step (KR20060112383A).

Historically, watercolor paints have mixed pigments with a liquid vehicle containing gum arabic, glycerin, honey, and/or ox-gall to make a paint that is moist or cake-like (Natural Pigments. Is There Anything New in Watercolors? https://www.naturalpigments.com/artist-materials/new-watercolor, accessed Nov. 13, 2023; Natural Earth Paint. Eco-friendly Paint Kits. https://naturalearthpaint.com/blogrecipe-professionalgrade-natural-watercolors/, accessed Nov. 13, 2023; Earth Pigments. Artist's Watercolor and Gouache. https://www.earthpigments.com/artists-watercolor-and-gouache/), accessed Nov. 13, 2023; Beebly's Watercolor Painting. History of Watercolor: Whereforth it came. https://watercolorpainting.com/blog/2015/09/14/history, accessed Nov. 13, 2023). To improve the texture (handling quality) of the paints over these early compositions, manufacturers have substituted or added other natural or synthetic organic ingredients: for example, starch or dextrin, polyvinylpyrrolidone, sorbitol, propylene glycol, and polyethylene glycol to improve the texture and handleability of the paints. Recipes widely available call for high concentrations of gum arabic (30%), high concentrations of glycerin (39%), and honey (18%). Our early efforts to create a paint using microalgal or cyanobacterial cells with a vehicle typical of that currently used for commercial watercolor paints failed to produce a paint that was photostable and that would dry in less than 24 hours when applied to paper. Thus, there is a need for a tailored water-based paint vehicle formula that will produce a paint with microalgal and cyanobacterial cells that is both color stable and will readily dry.

### Extraction

Isolated, partially purified or purified natural pigments derived from many biological sources can be used to make a paint. The biological material can be processed to make isolated pigments using a variety of methods, in some cases with added nutritional value. The sources of natural, living pigments include fruits, legumes, spices, vegetables, cyanobacteria, macroalgae and/or microalgae. For example, pigments are leached from plant roots (turmeric) or plant leaves to make paints and dyes. For another example, unicellular red algae were used to produce a water-soluble particulate coloring material (a phycobiliprotein) by grinding the microalgal cells. The methods for extracting the pigment from the biomass include pressure (screw press, hydraulic press), juicing (grinding, squeezing, or pressing), natural or synthetic solvent extraction, supercritical extraction, electrolysis, cavitation (ultrasonic treatment), heating, charring (pyrolysis), microwaving and/or distillation. These sources or methods may limit the colors available from natural sources; for example, black ink is produced by charring algal biomass (U.S. Pat. No. 11,739,219). Our invention does not use these steps, which add to costs and increase energy consumption and generate wastes.

One other method produced a paint using microalgal cells that required the color to slowly develop as the cells grew in the paint medium after their application to a substrate (US20150240093). In this application the cells must be living and multiplying at the time of use (and thus must be kept in a semi fluid state). Our invention does not require that the microalgal or cyanobacterial cells be alive at the time of application and upon application our paints remain in a dried state. The dried state is a key to enhancing the photostability of the paint.

### Additives

In a number of embodiments (e.g., SE545610C2), the colorants derived from living organisms are mixed with dispersants, surfactants, humectants, soaps, detergents, polymers, de- and anti-foamers, antioxidants, mordants, resins, oils/fats/fatty acids/lipids, water, saline solutions, decolorized whole microalgal cells, emulsifiers, and solvents to create a paint vehicle, binder, carrier, suspension, or emulsion to which pigments are added. These additives render hydrophobic pigments (e.g., chlorophyll, astaxanthin, beta-carotene) soluble in water (e.g., CPC A23L 5/44). However, the advantage of using whole cells in our invention is that hydrophobic pigments (e.g., chlorophyll, astaxanthin, beta-carotene) within the cells are rendered hydrophilic because the pigments are embedded in cell membranes that are surrounded by hydrophilic cell walls or cell components. Whole cells, which we use in our invention, also protect the pigments from degradation (e.g. due to photochemistry, oxidation, reduction, or hydrolysis). Further, the use of whole cells imparts a richness, dense opacity, and vibrancy to the paint which is not seen in paints made with purified water-soluble pigments (such as phycocyanin and phycoerythrin). Some of the components added for various purposes, —dispersants, surfactants, soaps, detergents, some polymers, saline solutions, and solvents (other than water)-promote the degradation of cell walls, which enables faster color deterioration (e.g., oxidation, fading, color change) of a paint, and these compounds are not needed, nor are they used, in our invention because intact cell structures protect the pigments. Our invention reduces the number of additives, thus reducing the cost and reducing any potential environmental impact that the production of these chemicals might have.

Although most applications use purified or partially purified pigments as a colorant, whole cells are used in certain instances for purposes other than as colorants. For example, a patent application filed in Sweden (SE545610C2) uses decolored microalgal cells as a binder to make an ink. A color or pigment from another source is inserted into the decolorized microalgal cells to obtain the desired hue. Our invention does not require this extra step of replacing the original pigment, and consequently our invention lowers the cost and simplifies the procedure; both benefits that may reduce the environmental and energy impact brought about by energy-intensive (e.g., supercritical fluid, reflux, or Soxhlet extraction), decolorizing methods. In our invention, the original color of the pigments is retained within the original cell, reducing the number of steps and cost.

In short, our invention is novel in two aspects. First, it uses whole cells instead of isolated pigments as colorants. Making paints from microalgae and cyanobacteria ensures that they do not contain added toxic heavy metals and do not rely on mining of heavy metals around the globe. Making paints from microalgae and cyanobacteria ensures that they also do not contain potentially toxic or carcinogenic synthetic organic chemicals as pigments. Our paints do not comprise species known to make hepatotoxins or neurotoxins and screening for the presence of these compounds can be performed using well-recognized commercial techniques. Our paints using microalgae and cyanobacteria do not use organic solvents or a heating step. Our paints made with microalgae and cyanobacteria have an ultra-low carbon footprint and can be manufactured anywhere in the globe. Second, the WBPV comprises a water-based formula with one or more humectants (e.g., honey), one or more photostabilizers (e.g., glycerol), one or more thickeners (e.g., gum arabic), one or more metal chelators (e.g., EDTA), one or more buffers (e.g., PIPES), one or more antioxidants (e.g., vitamin C, vitamin C vitamers, which are compounds that perform the function of vitamin C), and one or more UV-absorbing compounds (e.g., vitamin B12, cobalamins). These additives are known and common components of paint vehicles. Our invention combines these in concentrations unique to each specific microbial strain to produce stable, easily handled paints. Our invention includes cobalamin, which absorbs UVA (e.g., cyanocobalamin, vitamin B12, λₘₐₓ=368 nanometers, Labmate. 2024. https://www.labmate-online.com/article/mass-spectrometry-and-spectroscopy/41/mettler-toledo-gmbh-analytical/using-uvvis-spectroscopy-for-different-types-of-vitamin-b12-analysis/2324. Accessed Feb. 9, 2024), to protect against light-induced chemical breakdown of the pigments. This is a new component not reported in other natural, organic paints and, although it may be used as a nutritional supplement, not used in food additives to prevent color loss. The addition of cobalamin as a UVA shield is novel among inventions of colorant formulations. Our invention uses whole microalgal or cyanobacterial cells, which have advantages described above, and optimizes the formula for a given cell consortium, while reducing the number of steps necessary to make a natural, organic water-based paint.

### SUMMARY OF THE INVENTION

This invention embodies a water-based paint vehicle and method for making a photostable, colorfast paint from said vehicle and pigmented microalgal and cyanobacterial cells or their leachates. Because diverse species can be used to make paints, our paint colors span the entire wavelength spectrum of the color palette. The paint is used to make colorful art (FIG. 1). The cells are grown under progressively increasing light intensity to achieve the desired color, harvested to form a cell pellet, washed with distilled water to remove the residual culture medium, resuspended in the water-based paint vehicle, and dehydrated to a colored paint cake or powder. In some embodiments, the cells are exposed to distilled water or diluted water-based paint vehicle for several days at reduced temperature, which may leach the pigment from the cells. The resulting two-component suspension (cells and pigments) may be, but does not need to be, centrifuged or filtered to remove the depleted cell biomass; the paint may be used with both cells and leachate present and doing so imparts a richer texture and greater opacity than if the cells are removed. Water-based paint vehicle is added, and the colored solution is dehydrated to form a paint cake or powder. To use the invention, the paint cake or powder is rehydrated in water and painted onto a surface with a brush. The preferred surface is paper, but canvas, wood, metal, plastic, cloth, etc., may be used.

### BRIEF DESCRIPTION OF DRAWINGS

FIG. 1. Colorful art created with the invention.

FIG. 2. Table of examples of poorly characterized or uncharacterized microalgae with desired pigments that make photostable paints.

FIG. 3. Table of examples of poorly characterized or uncharacterized cyanobacteria with desired pigments that make photostable paints. UMPCCC numbered strains have 16S rRNA sequences deposited in GenBank.

FIG. 4. Paint made with cultured microalgal cells in either water only or in water-based paint vehicle. The left image shows the paints immediately after application to the surface. The right images show the corresponding paints, after exposure to light for 12 days. 1. Paint made with water only, 2. Paint made with water-based paint vehicle. Clumping, i.e. aggregation of cells, occurs in the absence of the water-based paint vehicle (top) for some strains. The water-based paint vehicle is necessary to prevent clumping.

FIG. 5. Effect of formula on color fastnesses of paint made with cultured microalgal cells and a water-based paint vehicle that may lack multivalent organic acids. The left image shows the paint made with microalgal cells and a water-based paint vehicle formula immediately after application to the surface. The right images show the corresponding paints after exposure to light for 17 days: 1. Water-based paint formula without citrate. 2. Water-based paint formula with 10×10⁻³ M citrate. 3. Water-based paint formula with 50×10⁻³ M citrate. The color changes in the absence or at lower concentrations of citrate (10×10⁻³ M) but is stable in the presence of higher concentrations of citrate (50×10⁻³ M). Citrate is necessary in the water-based paint vehicle to prevent color change (fading, changing color). Other derivatives of multivalent organic anions provide the same protection against color change.

FIG. 6. Effect of formula on color fastnesses of paint made with cultured microalgal cells and a water-based paint vehicle that may lack specific components. The left image shows the paint made with microalgal cells and a water-based paint vehicle formula immediately after application to the surface. The right images show the corresponding paints after exposure to light for 12 days: 1. Paint made with water-based paint vehicle. 2. Paint made with water-based paint vehicle without glycerin. 3. Paint made with water-based paint vehicle without artificial honey. 4. Paint made with water-based paint vehicle without gum arabic. The color does not change in the complete water-based paint vehicle formula (1), when the formula lacks glycerin (2), nor when the formula lacks gum arabic (4) for some strains. The color changes in the absence of artificial honey in the water-based paint vehicle (3). A humectant, of which artificial honey is the preferred type, is necessary in the formula to prevent color change (fading, loss, change in color).

FIG. 7. Effect of formula on stability after multiple rehydrations in paint made with cultured microalgal cells and a water-based paint vehicle that may lack specific components. The two left columns show the paint immediately after application to the surface and after exposure of that surface to light for at least 12 days. The two right columns show the paint applied to the surface after 10 rehydration steps without exposure to light and upon exposure to light for at least 12 days. 1. Water-based paint vehicle without citrate, K-sorbate, and vitamin C added. 2. Water-based paint vehicle with citrate but without K-sorbate and vitamin C. 3. Water-based paint vehicle with K-sorbate but without citrate and vitamin C. 4. Water-based paint vehicle with vitamin C but without citrate and K-sorbate. Top row shows microalgal cells with water-based paint vehicle lacking vitamin C, as an example of a vitamin C-type vitamer (compound that performs the function of vitamin C). The color changes under both dark (left pairs) and light (right pairs) conditions after 10 rehydrations. Bottom row shows microalgal cells with water-based paint vehicle containing vitamin C before and after 10 rehydrations. The color does not change under either dark (left pairs) or light (right pairs) conditions after 10 rehydrations. Vitamin C or its vitamers are necessary in the formula to prevent color change (fading, loss, change in color) for some types of microalgal cells.

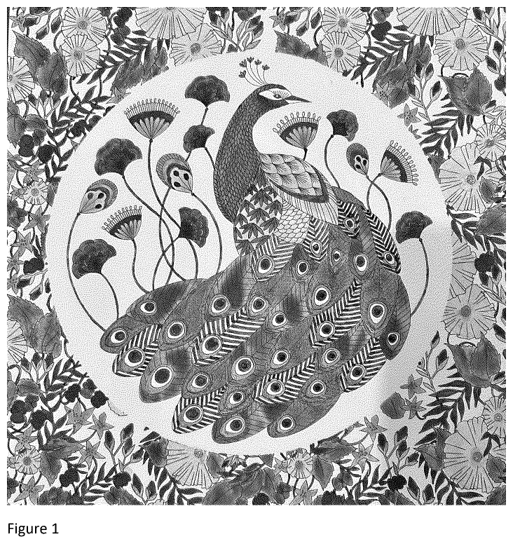

### DETAILED DESCRIPTION OF THE INVENTION

In the following detailed description, the paints according to the teachings for this application in the form of coloring agents and formula for a water-based paint vehicle (WBPV) will be described. This invention describes a lightfast, water-based paint where the pigment is derived from pigmented cells of algae or cyanobacteria suspended in a WBPV. The WBPV comprises a mixture of organic compounds including combinations with and without glycerol, a carbohydrate (such as natural honey, artificial honey, glucose, or sucrose), a polysaccharide (such as gum arabic, guar gum, or xanthan gum), a metal chelator and mold inhibitor (such as EDTA or NTA), a pH buffer (such as PIPES, Tris, or HEPES), sodium or potassium citrate, vitamin B12, and with or without vitamin C. The WBPV comprises GRAS (Generally Regarded As Safe) food-safe materials, and the pigments are made using microalgal or cyanobacterial cells that are grown phototrophically or organotrophically. When the cells containing the pigments are generated through the process of CO₂-fixation, the resulting paints have an ultra-low carbon footprint. The paints can be manufactured anywhere with access to clean water and power. The liquid paints are then dried for storage and can be rehydrated to a suspension to be used as a paint at a later date. The rehydrated suspension is then painted onto a surface (such as paper) and allowed to dry, creating a film that covers the surface material.

### Composition of the Algae

Paints using microalgal cells as the pigment can be generated using known commercial strains of algae and well-established methods for cultivating the strains. Commercial strains of algae include (but are not exclusive to) Porphyridium (a red microalga), Cyanophora paradoxa (a glaucophyte microalga), and Chlorella and Haematococcus spp. (both green microalgae). Porphyridium makes the pigment phycoerythrin, and Haematococcus makes ketocarotenoid pigments such as astaxanthin. Cyanophora and Cyanidium (aka Cyanidioschyzon, Galdieria) make the pigment phycocyanin. Chlorella species make pigments such as chlorophyll, beta-carotene, and astaxanthin. The microalgal cells can be grown autotrophically (using CO₂ as a carbon source) or organotrophically (using organic carbon as a carbon source). The microalgal cells can be grown phototrophically (using light as an energy source) or heterotrophically (using organic carbon as an energy source).

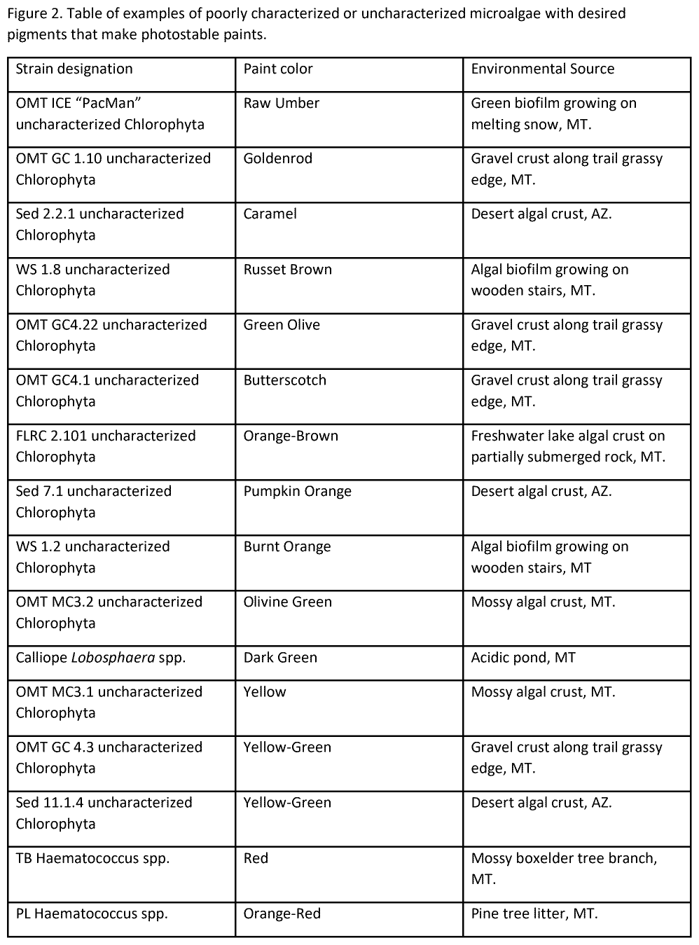

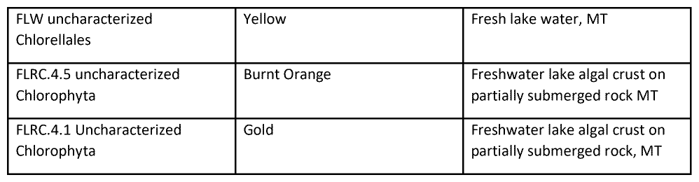

Paints using microalgal cells as pigments can also be generated using poorly characterized or uncharacterized strains of microalgae that synthesize desired pigments (FIG. 2).

### Composition of the Cyanobacteria

Paints using cyanobacteria as pigments can be generated using known commercial strains of cyanobacteria. Commercial strains include (but are not exclusive to) Arthrospira platensis (aka Spirulina, which makes the pigment phycocyanin). The cyanobacterial cells can be grown autotrophically (using CO₂ as a carbon source) or organotrophically (using organic carbon as a carbon source). The cyanobacterial cells can be grown phototrophically (using light as an energy source) or heterotrophically (using organic carbon as an energy source).

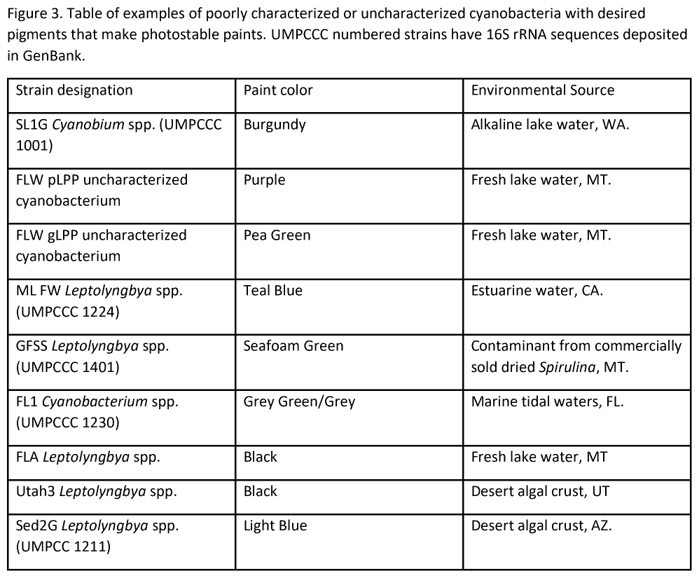

Paints using cyanobacteria as pigments can also be generated using poorly characterized or uncharacterized strains of cyanobacteria that synthesize desired pigments (FIG. 3).

### Composition of Alternative Pigmented Organisms

Alternatively, pigmented organisms that could be used to make a lightfast paint include macroalgae, naturally occurring pigmented microorganisms (e.g. Proteobacteria, Pseudomonadota, Heliobacteria, Halobacteria), genetically modified photosynthetic microorganisms, non-photosynthetic organisms (e.g., yeast or fungi), and genetically engineered or genetically modified organisms (e.g., Escherichia coli or Saccharomyces cerevisiae modified to produce pigments).

### Conditions for Growing Organisms

Growth conditions were optimized using standard laboratory procedures that someone skilled in the art would use to enhance the production of biomass and pigments.

### Lightfastness/Colorfastness/Photostability

Commercially available paint vehicles used for acrylic paints did not result in photostable paints using microalgae or cyanobacteria. Paint vehicle composition used for traditional oil paints also do not result in photostable paints using microalgae or cyanobacteria, and the pigments were found to leach from the cells in the paint onto and into the paper or canvas. Paint vehicle compositions used for traditional watercolor paints also did not result in photostable paints using algae or cyanobacteria. In order to discover an aqueous paint vehicle that could render microalgal and cyanobacterial paints photostable, the chemical formula of the paint vehicle was explored and optimized through experiments involving the iterative addition and subtraction of a variety of chemical components followed by photostability testing of each paint/vehicle composition. The concentration of each component was also varied followed by photostability tests.

Lightfastness/Colorfastness/Photostability of the paint was measured by preparing a paint sample and then painting the sample (approx. 50 microliters) onto watercolor paper or mixed media artist paper, allowing the paint to dry overnight, and then exposing the paint to direct sunlight for at least 12 continuous days. Photostability was further evaluated by rehydrating and dehydrating the paint 10 times each day for 10 successive days, painting a sample onto paper, allowing the paint to dry overnight, and then exposing the paint to direct sunlight for at least 12 continuous days. Photostable paints were determined to be those that did not fade after exposure to direct sunlight and after multiple successive rehydration/dehydration cycles. Further, prolonged exposure to light (LED, fluorescent room lights, and indirect sunlight) for more than a year does not lead to photobleaching of the dried paints.

### Formula of the Water-Based Paint Vehicle and Ranges of Concentrations

The WBPV formula yields a photostable paint for a wide variety of colors using microalgal and cyanobacterial cells as natural pigments. The preferred WBPV composition, which leads to a photostable paint for many cyanobacterial and microalgal strains tested, includes the following:

Natural or artificial honey: 19% honey is preferred, ranging from 0-33%. Artificial honey is preferred over natural honey. Alternatively, glucose, fructose, and/or sucrose or any combination of honey, glucose, fructose, and/or sucrose may also be used.

Glycerol: 7.2% preferred, ranging from 0-25%. Alternatively, xylitol and/or erythritol maybe used in place of, or in addition to, glycerol for some strains to make a photostable paint.

Gum arabic (also known as acacia gum, acacia fiber, Senegal gum, gum sudani): 3.4% preferred, ranging from 0%-9%. Alternatively, guar gum or xanthan gum could be used. Any combination of gum arabic, guar gum, and/or xanthan gum may also be used.

EDTA: (ethylenediamine tetraacetic acid; a metal chelator) 5 mM preferred, ranging from 0-20 mM. Alternatively, NTA (nitrilotriacetic acid) may be used alone or in combination with EDTA.

PIPES: 50 mM preferred, ranging from 0-100 mM. Alternatively, Tris and/or HEPES buffers may be used alone or in combination with PIPES.

Sodium or potassium citrate: 50 mM preferred, ranging from 0-100 mM. Alternatively, other multivalent organic anions may be used, alone or in combination with citrate.

Vitamin C: 0.6% solution preferred, ranging from 0-2%. Alternatively, other vitamin C vitamers may be used alone or in combination with vitamin C.

Cobalamin (such as vitamin B12): 0.0005% solution preferred, ranging from 0-0.005%.

### Examples of Addition and Subtraction of Components from the WBPV

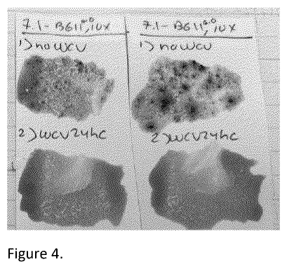

Example 1. Creating a paint by suspending microalgal or cyanobacterial cells in water (with no other aqueous components) resulted in a paint that clumped (FIG. 4). The resulting dried paint curled, clumped, and did not dry into a uniform film of paint. In addition, all paints made in this manner were not photostable over short to intermediate lengths of time (on the order of weeks to months) under ambient room lighting. Because of these properties, paints made by mixing water and cells were collectively undesirable.

Example 2. Addition of EDTA and PIPES to the WBPV improved the photostability of many paints and did not result in the decrease of photostability for any of the paints tested. The buffer (PIPES) and citrate helped buffer the acidic pH of the gum arabic solution to near neutral pH.

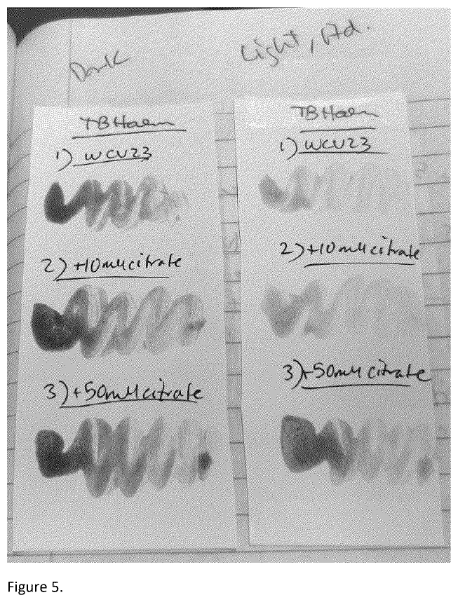

Example 3. Addition of citrate to the WBPV improved the photostability of all paints and did not result in the decrease of photostability for any of the paints tested. (FIG. 5)

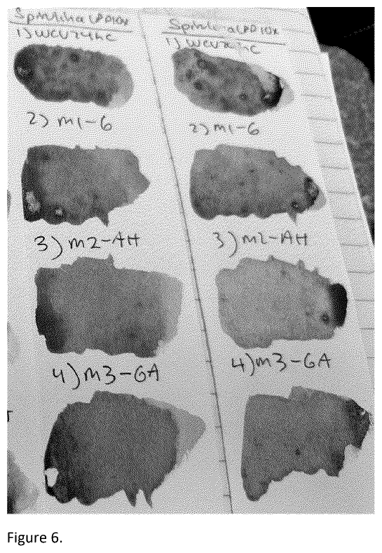

Example 4. Omission of glycerol, honey, or gum arabic from the WBPV sometimes decreased the photostability of some paints, as follows: Omission of any one of these had no effect on paint made using Calli Lobosphaera or OMT GC 4.22. Omission of artificial honey decreased the photostability of paint made with TB Haematococcus and ML FW Leptolyngbya. Omission of artificial honey or gum arabic reduced the photostability of paint made with GFSS Leptolyngbya and Sed2G Leptolyngbya. Omission of artificial honey resulted in a clumping paint for the Sed 7.1 strain. (FIG. 6)

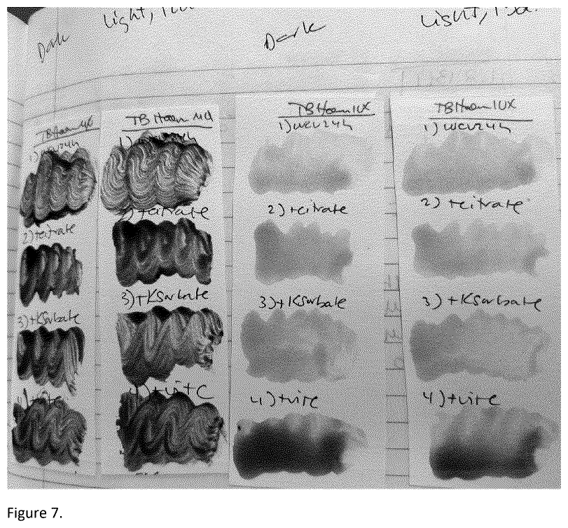

Example 5. Addition of vitamin C increased the photostability of some paints, such as paint made with TB Haematococcus, Calliope Lobosphaera, and OMT MC3.2. Addition of vitamin C, however, decreased the photostability of some paints, such as paint made with FL1 Cyanobacterium and GFSS Leptolyngbya or lead to an undesirable color change, such as paint made with Sed 7.1 and FLW Chlorella. (FIG. 7)

Example 6. Addition of vitamin B12 increased the photostability of some paints, such as paint made with TB Haematococcus and ML FW Leptolyngbya.

Example 7. Gum arabic could be readily exchanged with guar gum or xanthan gum. Paints that were equally stable with guar gum or xanthan gum include Calliope Lobosphaera, TB Haematococcus, OMT GC 4.22, ML FW Leptolyngbya, Sed2G Leptolyngbya, and GFSS Leptolyngbya.

Example 8. Glycerol could be readily exchanged with xylitol or erythritol. Paints that were equally stable with xylitol or erythritol include Calliope Lobosphaera, OMT GC 4.22, GFSS Leptolyngbya, and ML FW Leptolyngbya. Paint made with TB Haematococcus showed reduced photostability with xylitol or erythritol.

Example 9. Artificial honey could be readily exchanged with natural honey. A wide variety of paints made with artificial honey showed similar photostability as natural honey. Artificial honey was found to have a more consistent composition and coloration than natural honey and is readily produced from granulated sugar (sucrose).

### Making the Paint

To make the paints, microalgal or cyanobacterial cells from cultures are harvested from the culture medium by gravitational settling, centrifugation, or filtration (<0.2× micrometer (10⁻⁶ meter). The cells are washed with deionized or distilled water and then resuspended in the water-based paint vehicle to make a paint. The paints are then allowed to air dry as a cake, cone, pellet, powder, stick, or film and are stored in a dried state until they are ready for use.

To make some paints, harvested microalgal cells or cyanobacterial cells are permeabilized and water-soluble pigments (such as phycocyanin or phycoerythrin) are allowed to diffuse into the surrounding water or diluted paint vehicle. Permeabilization of the cells can be performed using cycles of freezing/thawing, cycles of dehydration/rehydration, or sonication. The cells are then either left in the paint or removed from the paint by centrifugation. The paint is then dried and stored in a dried state until it is ready for use.

To make some paints, purified water-soluble pigments, such as phycocyanin and phycoerythrin, are added to the WBPV. Commercial sources of phycocyanin and phycoerythrin are readily available. Water soluble pigments readily dissolve into the WBPV. The paint is then dried and stored in a dried state until it is ready for use.

### Usage of the Paint

The preferred usage of the paint is as a material to create colorful art and as an alternative to existing commercial watercolor paints that use heavy metals or synthetic organic dyes/chemicals.

To use the paint, the dried paint is reconstituted by adding water (deionized, distilled, bottled purified or tap water). The paint is fully mixed with a paint brush (or another implement) and is then painted onto a surface. The preferred surface is a paper surface, although other surfaces (glass, plastic, canvas, wood, metal, cloth, etc.) can also be painted. The paint is then allowed to dry (a few hours to overnight), leading to a photostable product that does not experience bleaching even upon long-term exposure (>12 months) to ambient room lighting. Any remaining paint is then allowed to dry until subsequent usage. The user can control the texture or shading of the paint by using an implement (brush or other) to move cells within a single paint stroke and can control the density of the paint pigments by adding more or less water to the paint. The paint we make has a thick creamy texture, however additional water or water color vehicle can be added to make a thinner paint suspension. Microalgal and cyanobacterial strains selected for use in a paint (examples of which are listed FIGS. 2 and 3) were chosen because they make a photostable paint that was robust to 10 serial rounds of hydration and dehydration.

Alternative uses of the paints include (but are not limited to) stenciling, stamping, writing/calligraphy, and silk screening.

Microalgal and cyanobacterial strains that are approved for food use (e.g., Arthrospira, Haematococcus, Chlorella, Chlamydomonas, and Dunaliella) could be used to make photostable, edible paints because all ingredients of the WBPV are GRAS. Wet paints could be used to color candy, chocolate, cakes, or cupcakes. Dried powdered paints could be also used to color ice cream, milk foam on hot and cold beverages, cakes, or cupcakes.

### Paints Changing Colors, Alteration of Paint Colors in Some Strains

Most paints show stable coloration as they are hydrated and dehydrated and as they are exposed to light over time. However, some exhibit small or significant changes in their coloration. Color change patterns of the paint are a unique characteristic of the species chosen to create the paint.

Some of the microalgal paints change color as the number of hydration/dehydration cycles increases, as follows: Paint made using Calliope Lobosphaera gradually changes from a dark green to a brownish green while paint made using OMT GC4.22 gradually changes from an olive green to an orange-brown (FIG. 4).

Some of the cyanobacterial paints show a change in color as phycocyanins and phycoerythrins are slowly released from the cell over time, as follows: Paint made using ML FW Leptolyngbya gradually changes from a grassy green to a teal blue. Paint made using Sed2G Leptolyngbya changes from a light green to a grayish blue. Paint made using FL1 Cyanobacterium changes from a dark green to a green/gray and then a gray. These and other known changes can be used to allow a painting to evolve.

1. A formula to make a paint comprising a water-based paint vehicle and pigmented microalgal or cyanobacterial cells (hereafter microbial cells).

2. A water-based paint vehicle (hereafter paint vehicle) according to claim 1, in which said vehicle contains water.

3. The paint vehicle according to claim 1, in which said vehicle contains a humectant, such as artificial or natural honey, glucose, fructose, and/or sucrose, singularly or in combination, in concentrations ranging from 0 to 33%; 19% preferred.

4. The paint vehicle according to claim 1, wherein said vehicle may contain a polyol (glycerol, or xylitol, or erythritol, or sorbitol, singularly or in combination) in concentrations ranging from 0 to 25%; 7.2% preferred.

5. The paint vehicle according to claim 1, wherein said vehicle contains a polysaccharide (gum arabic, guar gum, or xanthan gum, singularly or in combination) in concentrations ranging from 0 to 9%; 3.4% preferred.

6. The paint vehicle according to claim 1, wherein said vehicle is buffered to pH 7±1 by a pH buffer selected from the group consisting of 50×10⁻³ M (moles/liter) PIPES, Tris, or HEPES buffer singularly or in combination.

7. The paint vehicle according to claim 1, wherein said vehicle contains an antioxidant chelating agent, such as citrate, malate, or tartrate, singularly or in combination, in concentrations ranging from 0 to 100×10⁻³ M; 50×10⁻³ M preferred.

8. The paint vehicle according to claim 1, wherein said vehicle contains a metal chelator and mold inhibitor, such as EDTA, NTA, or DTPA, singularly or in combination, in concentrations ranging from 0 to 20×10⁻³ M; 5×10⁻³ M preferred.

9. The paint vehicle according to claim 1, wherein said vehicle may contain ascorbic acid (vitamin C), ascorbate, or vitamin-C vitamers, singularly or in combination, in concentrations ranging from 0 to 0.11 M; 0.034 M preferred.

10. The paint vehicle according to claim 1, wherein said vehicle may contain a cobalamin, such as vitamin B12, singularly or in combination, in concentrations ranging from 0 to 0.5 M; 0.05 M preferred.

11. The microbial cells according to claim 1, wherein said cells may contain microalgal cells.

12. The microbial cells according to claim 1, wherein said cells may contain cyanobacterial cells.

13. The microbial cells according to claim 1, wherein said cells may contain pigments derived from cells that have been permeabilized (for example by treatment, including but not limited to freeze-thawing or rehydration).

14. The microbial cells according to claim 1, wherein the microalgal cells may include commercial microalgal strains (e.g., Chlorella, Haematococcus, Porphyridium) or uncharacterized pigmented microalgal strains isolated from the natural environment.

15. The microbial cells according to claim 1, wherein the cyanobacterial cells may include commercial cyanobacterial strains (e.g. Spirulina, Arthrospira) or uncharacterized pigmented cyanobacterial strains isolated from the natural environment.

16. The process wherein the paint, according to claim 1, is made by creating a paint suspension by adding the paint vehicle to pelleted, compressed, or aggregated microbial cells in a ratio of 0.25-4 parts (17% to 80% v/v) paint vehicle to 1 part (20% to 83% v/v) microbial cells and, subsequently, is rendered light and shelf stable by dehydration into a paint cake or powder, which is rehydrated to the desired consistency prior to use to create art in the normal fashion.
